SEQUENCE LISTING (37 sequences)

SEQ ID NO: 1 - protein, 29 aa, Artificial Sequence
  synthetic consensus influenza hemagglutinin (HA) 2838-2866
BYPQYSEEARLKREEISGVKLESXGIYQI

SEQ ID NO: 2 - protein, 18 aa, Artificial Sequence
  synthetic consensus influenza M2e peptide 4120-4137, H5N1 MP Turkey/15/2006, H5N1 MP Egypt/902782/2006
VETPTRNEWECRCSDSSD

SEQ ID NO: 3 - protein, 89 aa, Artificial Sequence
  synthetic consensus influenza PB1-F2 1524-1612
XQGQDTPWTQSTEHTNIQKRGSGQXTQRLEHPNSTRLMDHYLRIMSPVXXHKQIVYWKQWLSLKNPTQGSLXTRVLKRWKLFNKQEWIN

SEQ ID NO: 4 - protein, 30 aa, Artificial Sequence
  synthetic consensus influenza hemagglutinin (HA) 2452-2481
KHLLSRINHFEKIQIIPKSSWSXHEASXGV

SEQ ID NO: 5 - protein, 51 aa, Artificial Sequence
  synthetic consensus influenza neuraminidase (NA) 3431-3481
QIGNMISIWVSHSIXTGNQXQXEXIXNXXXLTEXAVASVXLAGNSSLCPXX

SEQ ID NO: 6 - protein, 44 aa, Artificial Sequence
  synthetic consensus influenza hemagglutinin (HA) 2823-2866
KCDNECMESVRNGTYBYPQYSEEARLKREEISGVKLESXGIYQI

SEQ ID NO: 7 - protein, 320 aa, Artificial Sequence
  synthetic influenza hemagglutinin (HA) 2351-2670
DQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNS

SEQ ID NO: 8 - protein, 44 aa, Artificial Sequence
  synthetic specific influenza hemagglutinin (HA) 2823-2866
KCDNECMESVRNGTYDYPQYSEEARLKREEISGVKLESIGIYQI

SEQ ID NO: 9 - protein, 29 aa, Artificial Sequence
  synthetic specific influenza hemagglutinin (HA) 2838-2866, specific peptide from H5N1 HA2 domain, H5N1 HA Viet Nam/1203/2004
DYPQYSEEARLKREEISGVKLESIGIYQI

SEQ ID NO: 10 - protein, 23 aa, Artificial Sequence
  synthetic specific influenza M2e peptide 4115-4137, H5N1 M2 Viet Nam /1203/2004
SLLTEVETPTRNEWECRCSDSSD

SEQ ID NO: 11 - protein, 74 aa, Artificial Sequence
  synthetic specific influenza PB1-F2 1524-1598
EQGQDTPWTQSTEHTNIQKRGSGQQTQRLEHPNSTRLMDHYLRIMSPVGTHKQIVYWKQWLSLKNPTQGSLKTR

SEQ ID NO: 12 - protein, 30 aa, Artificial Sequence
  synthetic specific influenza hemagglutinin (HA) 2452-2481, H5N1 HA Viet Nam /1203/2004
KHLLSRINHFEKIQIIPKSSWSSHEASLGV

SEQ ID NO: 13 - protein, 51 aa, Artificial Sequence
  synthetic specific influenza neuraminidase (NA) 3431-3481, H5N1 NA Viet Nam/1203/2004
QIGNMISIWVSHSIHTGNQHQSEPISNTNFLTEKAVASVKLAGNSSLCPIN

SEQ ID NO: 14 - protein, 5 aa, Artificial Sequence
  synthetic specific M2e 4115-4137 amino terminus
SLLTE

SEQ ID NO: 15 - protein, 30 aa, Artificial Sequence
  synthetic influenza hemagglutinin (HA) 2452-2481 for H5N1 HA Indonesia/5/2005, Turkey/15/2006, A/Anhui/1/2005 and Egypt/902782/2006
KHLLSRINHFEKIQIIPKSSWSDHEASSGV

SEQ ID NO: 16 - protein, 30 aa, Artificial Sequence
  synthetic influenza hemagglutinin (HA) 2452-2481 for H1N1 HA New Caledonia/20/99
REQLSSVSSFERFEIFPKESSWPNHTVTGV

SEQ ID NO: 17 - protein, 27 aa, Artificial Sequence
  synthetic influenza hemagglutinin (HA) 2452-2481 for H3N2 HA California/07/2004
RSLVASSGTLEFNNESFNWTGVTQNGT

SEQ ID NO: 18 - protein, 29 aa, Artificial Sequence
  synthetic influenza hemagglutinin (HA) 2838-2866 for H5N1 HA Indonesia/5/2005
NYPQYSEEARLKREEISGVKLESIGTYQI

SEQ ID NO: 19 - protein, 29 aa, Artificial Sequence
  synthetic influenza hemagglutinin (HA) 2838-2866 for H5N1 HA Turkey/15/2006, A/Anhui/1/2005 and Egypt/902782/2006
DYPQYSEEARLKREEISGVKLESIGTYQI

SEQ ID NO: 20 - protein, 29 aa, Artificial Sequence
  synthetic influenza hemagglutinin (HA) 2838-2866 for H1N1 HA New Caledonia/20/99
DYPKYSEESKLNREKIDGVKLESMGVYQI

SEQ ID NO: 21 - protein, 29 aa, Artificial Sequence
  synthetic influenza hemagglutinin (HA) 2838-2866 for H3N2 HA California/07/2004
DHDVYRDEALNNRFQIKGVELKSGYKDWI

SEQ ID NO: 22 - protein, 51 aa, Artificial Sequence
  synthetic influenza neuraminidase (NA) 3431-3481 for H5N1 NA Indonesia/5/2005
QIGNMISIWVSHSIQTGNQHQAESISNTNPLTEKAVASVTLAGNSSLCPIR

SEQ ID NO: 23 - protein, 51 aa, Artificial Sequence
  synthetic influenza neuraminidase (NA) 3431-3481 for H5N1 NA Turkey/15/2006
QIGNMISIWVSHSIQTGNQRQAEPISNTKFLTEKAVASVTLAGNSSLCPIS

SEQ ID NO: 24 - protein, 51 aa, Artificial Sequence
  synthetic influenza neuraminidase (NA) 3431-3481 for H5N1 NA Anhui/1/2005
QIGNMISIWVSHSIQTGNQHQAEPIRNANFLTENAVASVTLAGNSSLCPVR

SEQ ID NO: 25 - protein, 51 aa, Artificial Sequence
  synthetic influenza neuraminidase (NA) 3431-3481 for H5N1 NA Egypt/902782/2006
QIGNMISIWVSHSIQTGNQCQAEPISNTKFLTEKAVASVTLAGNSSLCPIS

SEQ ID NO: 26 - protein, 51 aa, Artificial Sequence
  synthetic influenza neuraminidase (NA) 3431-3481 for H5N1 NA New Caledonia/20/1999
QIGNIISIWASHSIQTGSQNHTGVCNQRIITYENSTWVNHTYVNINNTNVV

SEQ ID NO: 27 - protein, 51 aa, Artificial Sequence
  synthetic influenza neuraminidase (NA) 3431-3481 for H3N2 NA California/07/2004
QIAILITTVTLHFKQYEFNSPPNNQVMLCEPTIIERNITEIVYLTNTTIEK

SEQ ID NO: 28 - protein, 89 aa, Artificial Sequence
  synthetic specific influenza PB1-F2 1524-1612
EQGQDTPWTQSTEHTNIQKRGSGQQTQRLEHPNSTRLMDHYLRIMSPVGTHKQIVYWKQWLSLKNPTQGSLKTRVLKRWKLFNKQEWIN

SEQ ID NO: 29 - protein, 74 aa, Artificial Sequence
  synthetic influenza PB1-F2 1524-1598 for H5N1 PB1-F2 Indonesia/5/2005
EQGQDTPWTQSTEHTNIQKRGSGQQTQRLEHPNSTRLMDHYLRIMSPVGMHKQIVYWKQWLSLKNPTQGSLKTR

SEQ ID NO: 30 - protein, 74 aa, Artificial Sequence
  synthetic influenza PB1-F2 1524-1598 for H5N1 PB1-F2 Turkey/15/2006
GQGQDTPWTQSTEHTNIQKRGSGQQTQRLEHPNSTRLMDHYLRIMSPVVMHKQIVYWKQWLSLKNPTQGSLKTR

SEQ ID NO: 31 - protein, 74 aa, Artificial Sequence
  synthetic influenza PB1-F2 1524-1598 for H5N1 PB1-F2 Egypt/902782/2006
GQGQDTPWTQSTEHTNIQKRGSGQKTQRLEHPNSTRLMDHYLRIMSPVVMHKQIVYWKQWLSLKNPTQGSLETR

SEQ ID NO: 32 - protein, 56 aa, Artificial Sequence
  synthetic influenza PB1-F2 1524-1598 for H1N1 PB1-F2 New Caledonia/20/1
GQEQGTPWIQSTGHISTQKEEDGQKIPKLEHRNSTQLMGHYQKTMNQVAMPKQIVY

SEQ ID NO: 33 - protein, 74 aa, Artificial Sequence
  synthetic influenza PB1-F2 1524-1598 for H3N2 PB1-F2 California/07/2004
EQEQGTPWTQSTEHTNIQRRGSGRQIQKLGHPNSTQLMDHYLRIMSRVDMHKQTVSWRLWPSLKNPTQVSLKTR

SEQ ID NO: 34 - protein, 72 aa, Artificial Sequence
  synthetic influenza PB1-F2 1524-1598 for H1N1 PB1-F2 Mexico/4486/2009 and California/04/2009
EQEQDTPWTQTEHTNTQKRESGRQTQRLVHPSSTRLMDHYLRIMNQVGMHKQTVFRLWLSLKNPTQEYLRIH

SEQ ID NO: 35 - protein, 23 aa, Artificial Sequence
  synthetic influenza M2e 4115-4138 for H1N1 M2 New Caledonia/20/1999 and H3N2 M2 California/07/2004 Indonesia/5/2005
SLLTEVETPIRNEWGCRCNDSSD

SEQ ID NO: 36 - protein, 23 aa, Artificial Sequence
  synthetic influenza M2e 4115-4138 for H1N1 M2 A/Mexico/4486/2009
SLLTEVETPTRSEWECRCSDSSD

SEQ ID NO: 37 - protein, 20 aa, Artificial Sequence
  synthetic influenza M2e 4115-4138 for H1N1 M2 A/California/04/2009
TEVETPTRSEWECRCSDSSD

(19) United States
(12) Patent Application Publication
(10) Pub. No.: US 2013/0137083 A1
(43) Pub. Date: May 30, 2013

(54) Detection of H5N1 Influenza Infection

(71) Applicants: Surender Khurana, Clarksburg, MD (US); Hana Golding, Rockville, MD (US)

(72) Inventors: Surender Khurana, Clarksburg, MD (US); Hana Golding, Rockville, MD (US)

(73) Assignees: National Institues of Health a government agency; The Goverment of the United States of Americaas Represented by the Secretary of the Department of Health and Human Services, Rockville, MD (US)

(21) Appl. No.: 13/641,687

(22) PCT Filed: Apr. 14, 2011
(86) PCT No.: PCT/US2011/032555

§ 371 Date: Feb. 14, 2013

Related U.S. Application Data
(60) Provisional application No. 61/325,073, filed on Apr. 16, 2010.

(51) Int. Cl. G01N 33/68

(52) U.S. Cl. CPC G01N 33/6893
USPC 435/5

(57) ABSTRACT

A combination of H5N1 influenza peptides that provide for H5N1 diagnosis with a high level of sensitivity and specificity is described.

### CROSS-REFERENCES TO RELATED APPLICATIONS

The present application claims the benefit of U.S. Provisional Application No. 61/325,073, filed on Apr. 16, 2010, which is incorporated herein by reference.

### BACKGROUND OF THE INVENTION

The recent spread of highly pathogenic H5N1 avian influenza viruses (AIV) among poultry and transmission of these viruses to humans raised concerns of a potential influenza pandemic. In preparation for such an event, world-wide efforts are under way to test and stockpile preventive vaccines, antiviral drugs, and passive immune therapies.

While some H5N1 peptides have been identified for diagnosis, an assay that has a sufficient sensitivity and especially specificity (i.e., low false positive rate) has not been described to the inventor's knowledge. In view of the close relationship of H5N1 to other influenza viruses, and the prevalence of seasonal flu, and therefore antibodies against such flu viruses, it is difficult to identify H5N1 peptides that are immunogenic and thus generate antibodies in infected individuals, and that do not significantly cross-react with antibodies generated in individuals infected with non-H5N1 influenza viruses.

### BRIEF SUMMARY OF THE INVENTION

As described below, the inventors have discovered a set of H5N1 peptides that are extremely useful in combination for detecting a wide range of different H5N1 strains from around the world, and yet do not have significant false positive activity, i.e., they do not generally cross-react with antibodies from individuals not infected with H5N1.

The present invention provides for methods for detecting the presence, absence, or quantity of H5N1 antibodies in a sample from an animal. In some embodiments, the method comprises,

(a) contacting the sample to:

a first polypeptide comprising a sequence at least 80% (e.g., at least 80%, 85%, 90%, or 95%) identical to SEQ ID NO:1, wherein the first polypeptide does not comprise more than 20 additional contiguous H5N1 Hemagglutinin amino acids on the amino terminus of SEQ ID NO:1 and the first polypeptide does not comprise more than five additional contiguous H5N1 Hemagglutinin amino acids on the carboxyl terminus of SEQ ID NO:1; and

a second polypeptide comprising a sequence at least 80% (e.g., at least 80%, 85%, 90%, or 95%) identical to SEQ ID NO:2, optionally with one or more amino acids of SLLTE (SEQ ID NO:14) linked to the amino terminus of SEQ ID NO:2, wherein the second polypeptide does not comprise more than five additional contiguous H5N1 M2e amino acids at either terminus; and

(b) detecting the presence, absence, or quantity of binding of at least one of the polypeptides to an antibody in the sample, thereby detecting the presence or absence of H5N1 antibodies in the sample.

In some embodiments, the contacting step (a) further comprises contacting the sample to a third polypeptide comprising a sequence at least 80% (e.g., at least 80%, 85%, 90%, or 95%) identical to SEQ ID NO:3, wherein the third polypeptide does not comprise more than five additional contiguous H5N1 PB 1-F2 amino acids at either terminus, and the detecting step (b) further comprises detecting the presence or absence of binding of the third polypeptide to an antibody in the sample.

In some embodiments, the contacting step (a) further comprises contacting the sample to a fourth polypeptide comprising a sequence at least 80% (e.g., at least 80%, 85%, 90%, or 95%) identical to SEQ ID NO:4, a fifth polypeptide comprising a sequence at least 80% (e.g., at least 80%, 85%, 90%, or 95%) identical to SEQ ID NO:5, or both the fourth and fifth polypeptide, wherein the fourth and fifth polypeptide do not comprise more than five additional contiguous H5N1 Hemagglutinin (HA) or Neuraminidase (NA) amino acids at either terminus, and the detecting step (b) further comprises detecting the presence or absence of binding of the fourth and/or the fifth polypeptide to an antibody in the sample.

In some embodiments, the first polypeptide comprises SEQ ID NO:1 and/or the second polypeptide comprises SEQ ID NO:2 and/or the third polypeptide comprises SEQ ID NO:3 and/or the fourth polypeptide comprises SEQ ID NO:4 and/or the fifth polypeptide comprises SEQ ID NO:5.

In some embodiments, the first polypeptide consists of SEQ ID NO:1 and/or the second polypeptide consists of SEQ ID NO:2 and/or the third polypeptide consists of SEQ ID NO:3 and/or the fourth polypeptide consists of SEQ ID NO:4 and/or the fifth polypeptide consists of SEQ ID NO:5.

In some embodiments, the second polypeptide comprises SEQ ID NO:8. In some embodiments, the first polypeptide comprises, or consists of, SEQ ID NO:9. In some embodiments, the second polypeptide comprises, or consists of, SEQ ID NO:10. In some embodiments, the third polypeptide comprises, or consists of, SEQ ID NO:11. In some embodiments, the fourth polypeptide comprises, or consists of, SEQ ID NO:12. In some embodiments, the fifth polypeptide comprises, or consists of, SEQ ID NO:13.

In some embodiments, the sample is from a human. In some embodiments, the sample is from a non-human animal. In some embodiments, the sample is from a bird.

The present invention also provides for kits for detecting the presence or absence of H5N1 antibodies in a sample. In some embodiments, the kit comprises:

a first polypeptide comprising a sequence at least 80% (e.g., at least 80%, 85%, 90%, or 95%) identical to SEQ ID NO:1, wherein the first polypeptide does not comprise more than 20 additional contiguous H5N1 Hemagglutinin amino acids on the amino terminus of SEQ ID NO:1 and the first polypeptide does not comprise more than five additional contiguous H5N1 Hemagglutinin amino acids on the carboxyl terminus of SEQ ID NO:1; and

a second polypeptide comprising a sequence at least 80% (e.g., at least 80%, 85%, 90%, or 95%) identical to SEQ ID NO:2, optionally with one or more amino acids of SLLTE (SEQ ID NO:14) linked to the amino terminus of SEQ ID NO:2, wherein the second polypeptide does not comprise more than five additional contiguous H5N1 M2e amino acids at either terminus,

and wherein the first and second polypeptides are linked to one or more solid supports.

In some embodiments, the kit further comprises contacting the sample to a third polypeptide comprising a sequence at least 80% (e.g., at least 80%, 85%, 90%, or 95%) identical to SEQ ID NO:3, wherein the third polypeptide does not comprise more than five additional contiguous H5N1 PB 1-F2 amino acids at either terminus.

In some embodiments, the kit further comprises contacting the sample to a fourth polypeptide comprising a sequence at least 80% (e.g., at least 80%, 85%, 90%, or 95%) identical to SEQ ID NO:4, a fifth polypeptide comprising a sequence at least 80% (e.g., at least 80%, 85%, 90%, or 95%) identical to SEQ ID NO:5, or both the fourth and fifth polypeptide, wherein the fourth and fifth polypeptide do not comprise more than five additional contiguous H5N1 Hemagglutinin or Neuraminidase amino acids at either terminus.

In some embodiments, the first polypeptide comprises SEQ ID NO:1 and/or the second polypeptide comprises SEQ ID NO:2 and/or the third polypeptide comprises SEQ ID NO:3 and/or the fourth polypeptide comprises SEQ ID NO:4 and/or the fifth polypeptide comprises SEQ ID NO:5.

In some embodiments, the first polypeptide consists of SEQ ID NO:1 and/or the second polypeptide consists of SEQ ID NO:2 and/or the third polypeptide consists of SEQ ID NO:3 and/or the fourth polypeptide consists of SEQ ID NO:4 and/or the fifth polypeptide consists of SEQ ID NO:5.

In some embodiments, the second polypeptide comprises SEQ ID NO:8. In some embodiments, the first polypeptide comprises, or consists of, SEQ ID NO:9. In some embodiments, the second polypeptide comprises, or consists of, SEQ ID NO:10. In some embodiments, the third polypeptide comprises, or consists of, SEQ ID NO:11. In some embodiments, the fourth polypeptide comprises, or consists of, SEQ ID NO:12. In some embodiments, the fifth polypeptide comprises, or consists of, SEQ ID NO:13.

In some embodiments, the first and second, and if included in the kit, the third, fourth and fifth polypeptides are linked to a solid surface.

### DEFINITIONS

The terms “polypeptide”, “peptide”, or “protein” are used interchangeably herein to designate a linear series of amino acid residues connected one to the other by peptide bonds between the alpha-amino and carboxy groups of adjacent residues. The amino acid residues are preferably in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and non-naturally-occurring amino acids.

The term “conservative substitution” is used in reference to proteins or peptides to reflect amino acid substitutions that do not substantially alter the activity (specificity or binding affinity) of the molecule. Typically conservative amino acid substitutions involve substitution one amino acid for another amino acid with similar chemical properties (e.g. charge or hydrophobicity). The following six groups each contain amino acids that are typical conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated.

The terms “isolated” or “substantially purified”, means a chemical composition that is essentially free of other cellular components. Such a composition can be in a homogeneous state although it can be in either a dry or aqueous solution. Purity and homogeneity are typically determined using analytical chemistry techniques such as polyacrylamide gel electrophoresis or high performance liquid chromatography or mass spectrometry. A protein which is the predominant species present in a preparation is substantially purified. Generally, a substantially purified or isolated protein will comprise more than 80% of all macromolecular species present in the preparation. In some embodiments, the protein is purified to represent greater than 90%, 95% of all macromolecular species present or is purified to essential homogeneity, wherein other macromolecular species are not detected by conventional techniques.

The terms “identical” or percent “identity,” in the context of two or more nucleic acids or polypeptide sequences, refer to two or more sequences or subsequences that are the same or have a specified percentage of nucleotides or amino acid residues that are the same (e.g., 60% identity, optionally 65%, 70%, 75%, 80%, 85%, 90%, 95%, 97% or 99% identity over a specified region), when compared and aligned for maximum correspondence over a comparison window, or designated region as measured using one of the following sequence comparison algorithms or by manual alignment and visual inspection. Alternatively, percent identity can be any integer from 25% to 100%. These definitions also refer to the complement of a test sequence.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window,” as used herein, includes reference to a segment of any one of the number of contiguous positions in which a sequence may be compared to a reference sequence of the same number of contiguous positions after the two sequences are optimally aligned. Methods of alignment of sequences for comparison are well known in the art.

Examples of an algorithm that is suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

### BRIEF DESCRIPTION OF THE DRAWINGS

FIG. 1 provides an alignment of various H5N1 peptide sequences of interest between different H5N1 isolates as well as alignment with some non-H5N1 influenza strains. FIG. 1(A) shows sequence conservation of selected peptides from H5N1, H1 and H3 strains (SEQ ID NOS:12, 12, 15, 15, 15, 15, 16, 17, 9, 9, 18, 19, 19, 19, 20, 21, 13, 13, 22, 23, 24, 25, 26 and 27, respectively). FIG. 1(B) shows alignment of peptide H5 PB1-F2 1524-1598 from H5N1, H1 and H3 strains (SEQ ID NOS:11, 11, 29, 30, 31, 32, 33, 34 and 34, respectively). FIG. 1(C) shows alignment of peptide H5 M2e from H5N1, H1 and H3 strains (SEQ ID NOS:10, 10, 2, 2, 35, 35, 36 and 37, respectively).

FIG. 2 provides summary data showing sensitivity of the assay for detection of antibodies from Vietnamese and Egyptian H5N1 survivors, family contacts and control (H5N1 unexposed) individuals in Egypt to various H5N1 peptides.

FIG. 3 provides summary data of detection of antibodies from individuals in Egypt that were exposed to H5N1 within the first two weeks post-infection to various H5N1 peptides.

FIG. 4 provides summary data showing specificity of the assay and non-reactivity of antibodies in sera from individuals vaccinated or infected with seasonal influenza as well as sera from additional H5N1 vaccinated individuals to various H5N1 peptides.

### DETAILED DESCRIPTION

### I. Introduction

The present invention is based, in part, on the discovery that combinations of particular H5N1 peptides are capable of specifically detecting anti-H5N1 antibodies, and thus current or past H5N1 infection in a large portion of H5N1-infected individuals without significant numbers of false positive results. Further, the peptide combinations described herein have been tested in H5N1-infected individuals from different parts of the world (e.g., Egypt, Vietnam) and accordingly have been determined to detect antibody response to different H5N1 variants. Thus, the combination of the particular peptides identified and selected by the inventors provide an excellent way to detect H5N1 exposure in individuals with a high level of specificity and sensitivity.

The present invention has the advantage of providing a less expensive alternative to current PCR-based assays that can only be conducted with nasopharyngeal washes of suspected infections up to several weeks post exposure. The serodiagnostic assay described herein can be used to detect exposure to H5N1 in blood samples in less than seven days and up to several years post-exposure. The serodiagnostic assay of the invention may also be able to detect seroconversion in individuals who come into contact with infected individuals but did not develop full blown influenza disease.

### II. Useful Peptide Combinations for Specifically Detecting H5N1

The inventors have found that detection in individuals of antibodies to a specific peptide from the H5N1 HA2 domain (HA 2838-2866; SEQ ID NO:9) is useful for detection of exposure to certain H5N1 strains. For example, all infected individuals in one trial in Vietnam were identified based on the presence of antibodies that bound SEQ ID NO:9. However, due to strain variation or at early time point post-H5N1 infection, some sera do not recognize SEQ ID NO:9. For example, fewer than half of the H5N1-infected individuals tested in Egypt had antibodies that bind to SEQ ID NO:9. The inventors have found surprisingly that detection of antibody response to SEQ ID NO:9 and a portion of H5N1 M2e (M2e 4115-4137; SEQ ID NO:10) complement each other such that nearly all H5N1-infected individuals tested could be identified as H5N1-positive using these two peptides in combination in one diagnostic assay. The remaining few individuals that did not test positive for antibodies that bound with SEQ ID NO:9 or SEQ ID NO:10 could be identified by screening for antibodies to at least one, two, or all three of a portion of H5N1 protein PB1-F2 (PB1-F2 1524-1598; SEQ ID NO:11), H5N1 HA1 (HA1 2452-2481; SEQ ID NO:12) and/or H5N1 NA (NA 3431-3481; SEQ ID NO:13). Accordingly, the present invention provides for detection of antibody response in individuals to at least H5N1 HA 2838-2866 and/or H5N1 M2e 4115-4137, and optionally at least one or more of H5N1 PB1-F2 1524-1598, H5N1 HA1 2452-2481 and/or H5N1 NA 3431-3481. In some embodiments, the polypeptides of the invention are isolated, purified, or both.

Notably, none of the above-described peptides (in contrast to many others tested) generated a single false-positive signal in those individuals tested. For example, individuals vaccinated with H5N1 vaccine, or following infection with H1N1 or H3N2 seasonal influenza did not generate antibodies directed to any of the above-described peptides. Therefore, the above-described peptides provide for an optimal assay that is (1) capable of detecting individuals who have been exposed to any of several different H5N1 variants but (2) can distinguish from exposure to other more common influenza viruses.

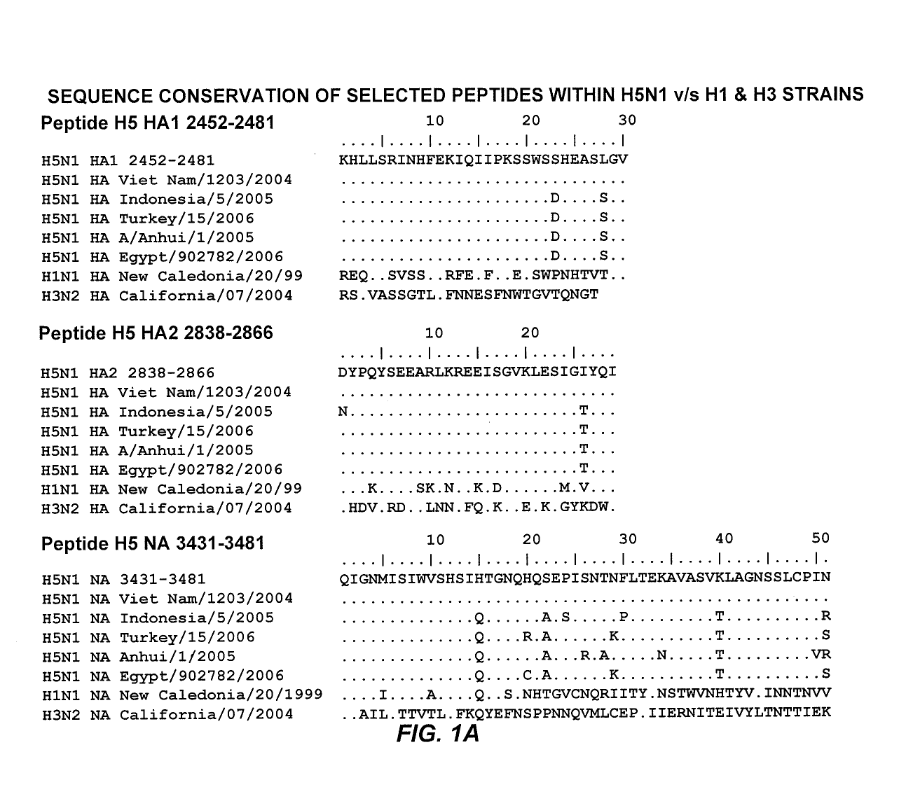

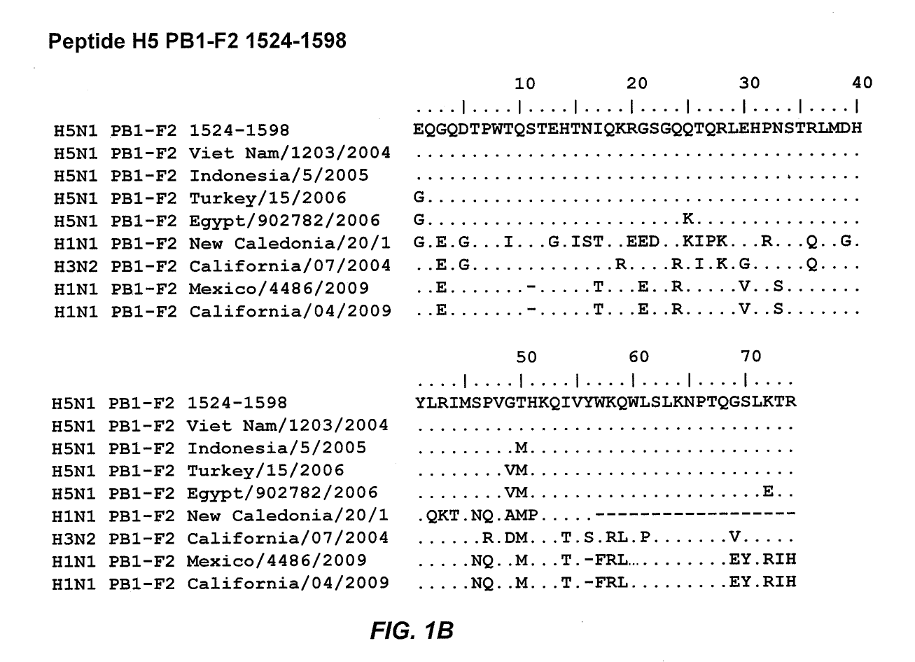

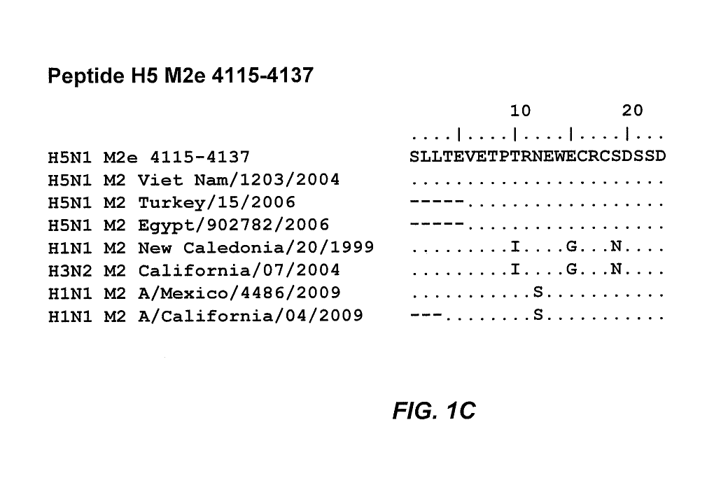

Alignments of several H5N1 variant sequences in the relevant regions are displayed in FIG. 1. The alignments demonstrate possible slight variants than can be used in the peptides of the invention. Accordingly, the invention provides for use of variants of the above-described peptide sequences in assays to detect H5N1 exposure. Exemplary variants include, e.g., peptides having one, two, three, four, or five amino acid variations from any of SEQ ID NO:9, SEQ ID NO:10, SEQ ID NO:11, SEQ ID NO:12, or SEQ ID NO:13 as determined by an alignment algorithms such as BLAST. In some embodiments, the assays of the invention employ a peptide at least 80%, 85%, 90%, or 95% identical to SEQ ID NO: 9 and a peptide at least 80%, 85%, 90%, or 95% identical to SEQ ID NO:10, optionally further including one or more sequence at least 80%, 85%, 90%, or 95% identical to SEQ ID NO:11, SEQ ID NO:12, or SEQ ID NO:13. Guidance in determining which amino acid residue may be inserted, substituted or deleted without adversely affecting the desired activity may be found by comparing the sequence of the polypeptide with that of homologous known protein sequences (see, e.g., FIG. 1) and minimizing the number of amino acid sequence changes made in regions of high homology. Further, in some embodiments, one, two, three, four, or five, or more conservative amino acid changes are made to H5N1 peptides of the invention, while maintaining desired antibody cross-reactivity. In addition, the crystal structure of many of the influenza virus proteins are known and can be accessed at the Protein Data Bank. Modeling of the effect of any amino acids changes on the structure can be determined by using available computer programs.

Moreover, in view of the alignments, in some embodiments, the polypeptide in the assay corresponding to HA 2838-2866 is (N/D)YPQYSEEARLKREEISGVKLES(I/T)GIYQI (SEQ ID NO:1), where alternative positions are indicated in parentheses. The alternate amino acids occur in different H5N1 variants a shown in FIG. 1. In some embodiments, the polypeptide corresponding to M2 4120-4137 is VETPTRNEWECRCSDSSD (SEQ ID NO:2), i.e., lacking the amino-terminal SLLTE (SEQ ID NO:14) in SEQ ID NO:10. As shown in FIG. 1, some of the variants of H5N1 do not have the SLLTE (SEQ ID NO:14) sequence that is present in the M2 4115-4137 region of H5N1 Vietnam 1203/2004 isolate.

In some embodiments, the polypeptide corresponding to PB 1-F2 (1524-1612) is (E/G)QGQDTPWTQSTEHTNIQKRGSGQ(Q/K)TQRLEHPNSTRLMDHYLRIMSPV(G/V) (T/M)HKQIVYWKQWLSLKNPTQGSL(K/E)TRVLKRWKLFNKQEWIN (SEQ ID NO:3). In some embodiments, the polypeptide corresponding to HA1 2452-2481 is KHLLSRINHFEKIQIIPKSSWS(S/D)HEAS(L/S)GV (SEQ ID NO:4). In some embodiments, the polypeptide corresponding to NA 3431-3481 is QIGNMISIWV SHSI(H/Q)TGNQ(H/R/C)Q(S/A)E(P/S)I(S/R)N(T/A)(N/K)(F/P)LTE(K/N)A VASV(K/T)LAGNSSLCP(I/V)(N/R/S) (SEQ ID NO:5).

While the above-described polypeptide sequences can comprise additional amino acids at the amino or carboxyl terminus, it is generally desirable that they not include additional contiguous or non-contiguous sequences from H5N1 to avoid generating longer polypeptide sequences that cross-react with antibodies directed against non-H5N1 influenza viruses. Generally, to maintain specificity of the assay, at most only a minimal number of additional adjacent contiguous H5N1 amino acids, if any, are added to the amino- or carboxyl-terminus, or both, of the diagnostic sequences described herein. Thus, in some embodiments five or fewer adjacent H5N1 amino acids (e.g., M2 4114-4137, or M2 4114-4142 instead of M2 4115-4137) are included at either the amino or carboxyl terminus, or both, without creating significant false positive cross-reactivity. However, the inventors have found that the amino terminus of SEQ ID NO:1 can be extended farther without generating significant false positive reactions. For example, the inventors have found that SEQ ID NO:6, which contains fifteen additional contiguous adjacent amino aids of H5N1 HA protein, does not cause cross-reactivity. Accordingly, in the case of SEQ ID NO:1, in some embodiments, twenty (e.g., 20, 15, 10, 5, etc.) or fewer adjacent H5N1 amino acids are included at the amino terminus of SEQ ID NO:1. Alternatively, or in addition, five or fewer adjacent H5N1 amino acids are included at the carboxyl terminus of SEQ ID NO:1 without creating significant false positive cross-reactivity.

In addition, in some embodiments, one or more polypeptide of the invention includes one or more additional heterologous non-H5N1 amino acid sequences at the N or C-terminus of the polypeptide. Heterologous sequences can be used, for example, to fuse the polypeptides to other molecules or surfaces, to improve expression and folding of the polypeptides during expression, or to include a protease cleavage site or other desirable property. Of course, the heterologous sequences can be screened for cross-reactivity to antibodies against non-H5N1 influenza strains or other undesirable cross-reactivity. Accordingly, in some of the embodiments of the invention, the polypeptides comprise, or consist of, for example, SEQ ID NO:1, SEQ ID NO:2, SEQ ID NO:3, SEQ ID NO:4, SEQ ID NO:5, SEQ ID NO:6, SEQ ID NO:8, SEQ ID NO:9, SEQ ID NO:10, SEQ ID NO:11, SEQ ID NO:12, or SEQ ID NO:13 (and optionally at five or fewer, e.g., 5, 4, 3, 2, 1 contiguous adjacent H5N1 amino acids) but do not include further H5N1 amino acid sequences of at least 10 amino acids long (with the exception of SEQ ID NO:1, which can include additional amino terminal amino acids as discussed above).

Polynucleotides encoding influenza polypeptides, recombinant vectors, and host cells containing the recombinant vectors, as well as methods of making such vectors and host cells by recombinant methods are useful to produce the polypeptides as described herein for use in diagnostic assays. The polynucleotides of the disclosure may be synthesized by chemical methods or prepared by techniques well known in the art. See, for example, Creighton, Proteins: Structures and Molecular Principles, W. H. Freeman & Co., New York, N.Y. (1983). Nucleotide sequences encoding the influenza polypeptides of the disclosure may be synthesized, and/or cloned, and expressed according to techniques well known to those of ordinary skill in the art. See, for example, Sambrook, et al., Molecular Cloning, A Laboratory Manual, Vols. 1-3, Cold Spring Harbor Press, Cold Spring Harbor, N.Y. (1989). In some embodiments, the polynucleotide sequences will be codon optimized for a particular host cell using standard methodologies. For example, the DNA construct encoding a H5N1 HA polypeptide can be codon optimized for expression in bacterial cells.

The polynucleotides encoding the polypeptides of the invention may be produced by standard recombinant methods known in the art, such as polymerase chain reaction (PCR) or reverse transcriptase PCR (Sambrook, et al., 1989, Molecular Cloning, A Laboratory Manual, Vols. 1-3, Cold Spring Harbor Press, Cold Spring Harbor, N.Y.), reverse engineering, or the DNA can be synthesized and optimized for expression in bacteria or eukaryotic cells. Primers can be prepared using the polynucleotide sequences that are available in publicly available databases. The polynucleotide constructs may be assembled from polymerase chain reaction cassettes sequentially cloned into a vector containing a selectable marker for propagation in a host. Such markers include but are not limited to dihydrofolate reductase or neomycin resistance for eukaryotic cell culture and tetracycline, ampicillin, or kanamycin resistance genes for culturing in E. coli and other bacteria.

Representative examples of appropriate hosts include, but are not limited to, bacterial cells such as E. coli, Streptomyces and Salmonella typherium, fungal cells such as yeast; insect cells such as Drosophilia S2 and Spodoptera Sf9, animal cells such as CHO, COS, and Bowes melanoma cells, and plant cells. Appropriate culture medium and conditions for the above-described host cells are known in the art.

The influenza polypeptide(s) can be recovered and purified from recombinant cell cultures by methods known in the art, including but not limited to ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography.

### III. Methods of Detection

The H5N1 peptide combinations described herein can be used in immunoassays for diagnosing or prognosing H5N1 infection or exposure in an animal, including but not limited to humans, animals and birds (e.g., poultry, chickens, ducks, geese, ferrets, horses, pigs, etc.). In some embodiments, the assays of the invention are used to distinguish between different subtypes of influenza virus infection, between vaccinated and infected subjects; and/or between different clades or variants of H5N1 influenza virus subtypes. These assays can be used in surveillance of emerging pandemics, and may be particularly useful in countries that do not have the ability to run PCR type assays.

The diagnostic methods of the invention can include, e.g., a method for determining the presence of a H5N1 infection in a subject comprising analyzing a biological sample to detect the presence of an antibody that specifically binds to one or more polypeptides as described herein, wherein the presence of the antibody is indicative of H5N1 infection. Immunoassays using the H5N1 peptide combinations provide a highly specific, sensitive and reproducible method for diagnosing H5N1 infections, in contrast to immunoassays that use fewer peptides or that use significantly more H5N1 sequence.

Immunodetection methods can include, but are not limited to, enzyme linked immunosorbent assay (ELISA), rapid immunoassay (including point-of-care tests, lateral flow, agglutination, latex beads linking assay, etc.), radioimmunoassay (RIA), immunoradiometric assay, fluoroimmunoassay, chemiluminescent assay, bioluminescent assay, solution based antigen-antibody interaction assays and Western blot to mention a few. The steps of various useful immunodetection methods have been described in the scientific literature. A variety of immunoassay formats may be used to detect antibodies specifically immunoreactive with a particular protein. For example, solid-phase ELISA immunoassays are routinely used to select antibodies specifically immunoreactive with a protein or carbohydrate. See Harlow and Lane (1988) Antibodies, A Laboratory Manual, Cold Spring Harbor Publications, New York, for a description of some exemplary immunoassay formats and conditions that can be used to determine specific immunoreactivity. Such assays may be a direct, indirect, competitive, or noncompetitive immunoassays, for example as described in the art (e.g., Oellerich, M. 1984. J. Clin. Chem. Clin. BioChem. 22: 895-904). Biological samples appropriate for such detection assays include, but are not limited to, tissue biopsy extracts, whole blood, plasma, serum, cerebrospinal fluid, pleural fluid, urine, saliva or other oral fluid and the like.

In some embodiments, serum or another sample from a human or non-human animal is reacted with a solid phase reagent having a combination of surface-bound recombinant polypeptides of the invention as an antigen. For example, one, two, three, four, five, or more of the polypeptides as described herein can be attached to a solid substrate such as a bead, ELISA plate, dipstick, or microarray. In some embodiments, the polypeptides include, e.g., polypeptides comprising or consisting of H5N1 HA 2838-2866 (e.g., SEQ ID NO:1 or SEQ ID NO:9) and H5N1 M2e 4115-4137 (e.g., SEQ ID NO:2 or SEQ ID NO:10) and little (e.g., fewer than five amino acids) or no other additional H5N1 contiguous sequence, or variants thereof as described herein, optionally in combination with polypeptides comprising or consisting of PB1-F2 1524-1598 (e.g., SEQ ID NO:11), H5N1 HA1 2452-2481 (e.g., SEQ ID NO:12) and/or H5N1 NA 3431-3481 (e.g., SEQ ID NO:13) and little (e.g., fewer than five amino acids) or no other additional H5N1 contiguous sequence, or variants thereof as described herein.

The solid surface reagent can be prepared by known techniques for attaching protein to solid support material. These attachment methods can include non-specific adsorption of the protein to the support or covalent attachment of the protein to a reactive group on the support. In some embodiments, after reaction of the antigen with an anti-H5N1 antibody from a biological sample, unbound sample components are removed by washing. Depending on the format of the assay, in some embodiments, the antigen-antibody complex is reacted with a secondary antibody, e.g. a labeled anti-human antibody. In other embodiments, a secondary antibody that specifically binds the bound antigen can be used as a secondary antibody and binding of the secondary antibody can then be detected and optionally quantified. Other assay formats are also available, for example as known in the art.

Any of a variety of detectable labels can be used and can be linked to different components of the assay depending on the configuration of the assay. In some embodiments, the label is an enzyme that is detected by incubating the solid support in the presence of a suitable fluorimetric or calorimetric reagent. Other detectable labels may also be used, such as fluorescent labels, radiolabels or colloidal gold, and the like.

In some embodiments, the serodiagnostic assay can detect exposure to H5N1 within less than 7 days and up to at least 4 years after infection. For example, the serodiagnostic assay can detect antibodies resulting from exposure to H5N1 within about one to 10 days and up to 1, 2, 3, 4 or more years post-infection. In some embodiments, the serodiagnostic assay can detect exposure to H5N1 within about 2 to 10 days, 3 to 10 days, 4 to 10 days, 5 to 10 days, 6 to 10 days, or 7 to 10 days post-infection. In other embodiments, serodiagnostic assay can detect exposure to H5N1 within about one to seven days post-infection, for example about 3 to 7 days, 4 to 7 days, 5 to 7 days, and 6 to 7 days post-infection. In other embodiments, the serodiagnostic assay can detect exposure to H5N1 one, two, three, four, five, six, seven, eight, nine, or 10 days or longer post-infection.

### IV. Kits

The H5N1 peptide combinations of the invention can be prepared in the form of a kit, alone, or in combinations with other reagents such as secondary antibodies, labels, label substrates, or other reagents for use in immunoassays. The kits of the invention can comprise any combination of two or more peptides described herein, optionally in a single or multiple containers. In some embodiments, one or more of the polypeptides are linked to a solid support (e.g., as part of an ELISA assay or other immunoassay kit).

In some embodiments, one, two, three, four, five, or more of the polypeptides as described herein, optionally linked to a solid support, are included in the kit. In some embodiments, the polypeptides in the kit include, e.g., polypeptides comprising or consisting of H5N1 HA 2838-2866 (e.g., SEQ ID NO:1 or SEQ ID NO:9) and H5N1 M2e 4115-4137 (e.g., SEQ ID NO:2 or SEQ ID NO:10) and little or no other H5N1 contiguous sequence, or variants thereof as described herein, optionally in combination with polypeptides comprising or consisting of PB1-F2 1524-1598 (e.g., SEQ ID NO:11), H5N1 HA1 2452-2481 (e.g., SEQ ID NO:12) and/or H5N1 NA 3431-3481 (e.g., SEQ ID NO:13) and little or no other H5N1 contiguous sequence, or variants thereof, e.g., as described herein.

### EXAMPLES

The following examples are offered to illustrate, but not to limit the claimed invention.

### Example 1

### H5N1 Diagnostic Peptides

We have developed an assay based on H5N1 peptides having the following characteristics:

I. A simple H5N1 serodiagnostic assay based on long-lasting, highly conserved (cross clades) antibody epitopes;
II. An assay having specificity with an emphasis on differentiating between exposure to seasonal influenza (H1N1, H3N2, B) vs. avian H5N1 influenza;
III. A serodiagnostic assay to distinguish between vaccine induced-antibodies and true exposure to H5N1 viruses.

To this end we identified immunodominant epitopes that reacted strongly with H5N1 convalescent sera but not with control sera from unexposed Vietnamese or with sera from US individuals with known titers against seasonal influenza. We focused on 5 peptides that: (a) are highly conserved among H5N1 clades and subtypes, (b) have high sequence diversity between seasonal vs. H5N1 influenza viruses, and (c) are not recognized by H5N1 post vaccination antibodies. As shown in FIG. 1 (A-C), the sequence for the selected peptides is >80% conserved among different H5N1 influenza strains. Few variations in the peptide sequence can be introduced to increase the reactivity of the peptides with infected sera from the homologous strain from different H5N1 Glade as shown in sequence alignment for these peptide sequences. For example, HA2 2838-2866 peptide might react better with post-H5N1 infected samples from Egypt, if it contains a T (threonine) in place of I (isoleucine) in the corresponding homologous H5N1 sequence from the Egypt viral strain.

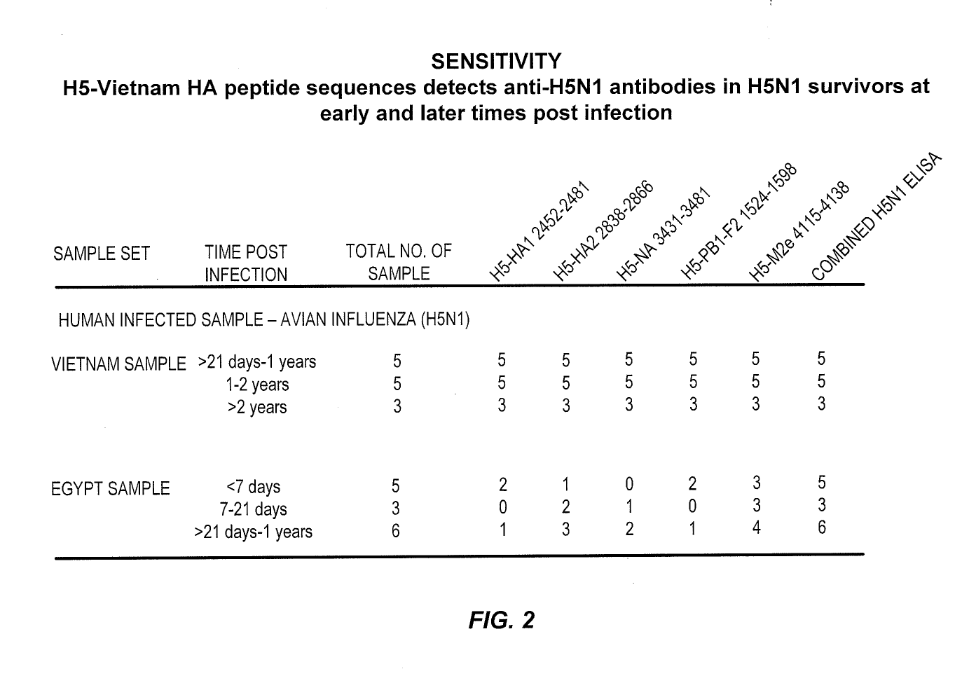

These peptides were tested with a large panel of human sera. The data generated is summarized in FIGS. 2-4, which demonstrate the sensitivity and specificity of these peptides in a serodiagnostic assay.

FIG. 2 shows that H5N1 peptide sequences detect anti-H5N1 antibodies in H5N1 exposed survivors at both early and later time point post infection. Convalescent serum samples from human who survived infection with H5N1 in Vietnam and Egypt collected acutely after infection and up to 4 years post-infection were analyzed for reactivity with the selected peptides. All post-H5N1 infection sera reacted with multiple H5N1 peptides and showed 100% sensitivity of the H5N1 serodiagnostic assay. Peptides having SEQ ID NO: 9, 10 or 11 showed the strongest reactivity with these sera.

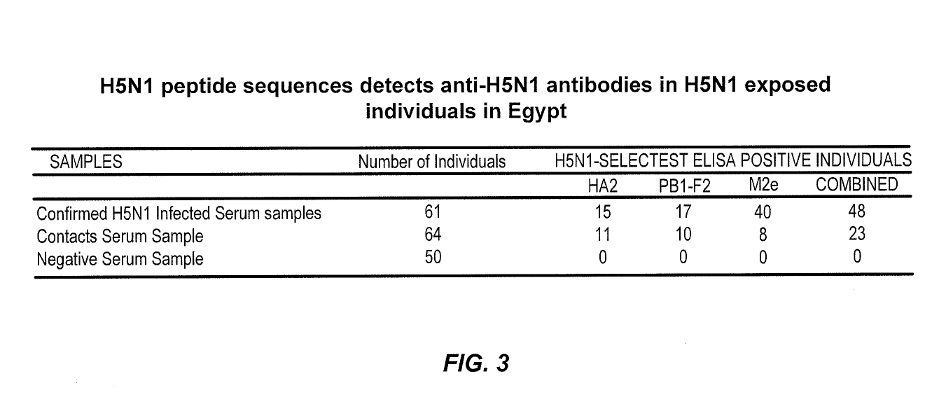

FIG. 3 summarizes the reactivity of large panel of sera from recent H5N1 infections in Egypt. Sera from 61 acutely H5N1-infected individuals after 1-10 days post-H5N1 infection was obtained and tested with the selected three H5N1 peptides that showed strongest reactivity in the first analysis in FIG. 2. All the sera from 48 confirmed H5N1 infected individuals that had sera collected within 4-10 days post-infection reacted in H5N1 peptide ELISA and were found to be seropositive. Thirteen of the H5N1 infected individuals that had sera collected within 1-3 days post-H5N1 infection did not react with our H5N1 peptides. So by this analysis our H5N1 serodiagnostic assay can detect antibodies after 3^{rd} day post-H5N1 infection. 36% of the sera from the individuals who came in contact with their confirmed H5N1-infected family members were observed to react to H5N1 peptides in our ELISA. This may be due to sub-clinical limited H5N1 exposure in these contact persons. None of the sera from 50 H5N1-unexposed individuals reacted with any of the H5N1 peptides. These sera were collected from individuals who visited hospital due the other infectious disease endemics in Egypt including tuberculosis, malaria, dengue fever, etc.

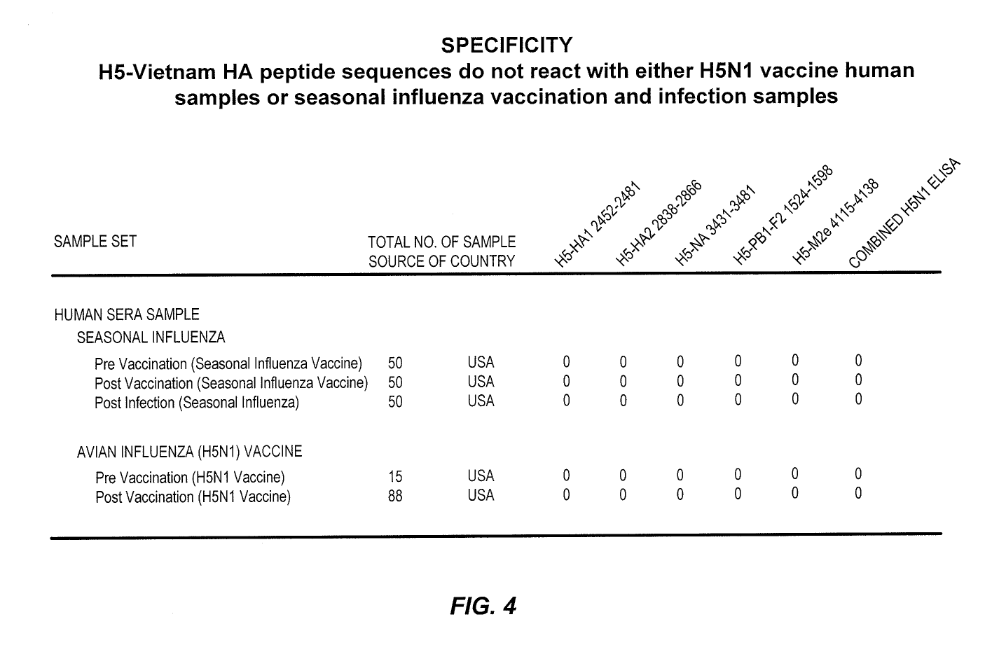

FIG. 4 summarizes the reactivity of a large panel of sera from Normal (Unexposed) samples and Avian (H5N1) Influenza vaccine recipients in US. None of 50 sera samples from either confirmed seasonal influenza post-vaccination or post-infection samples reacted with any of the H5N1 peptides. Evaluation of a large panel of post-H5N1 vaccination plasma samples from both A/Vietnam and A/Indonesia vaccine recipients, both without adjuvant and with adjuvant through sites participating in NIH sponsored vaccine trials, did not show any seropositivity with the selected five H5N1 peptides. This analysis demonstrates the specificity of these particular peptides for detecting H5N1 infection.

The H5N1 serodiagnostic assay as outlined above allows for a simple high throughput H5N1 sero-diagnostic assay for surveillance of general populations, including in previously vaccinated regions. It has the potential to shorten the time to diagnosis in the early stages of avian influenza outbreaks and facilitate early initiation of measurements to treat infected individuals and curtail human spread by various means including passive and active vaccinations.

### Example 2

### Analysis of H5N1 Infections in Vietnam

This example shows that the diagnostic peptides of the invention can detect 100% of the confirmed H5N1 infected individuals soon after infection.

Sera from 44 convalescent individuals who were recently infected in Vietnam was assayed as described above. As shown in Table 1, sera was collected from individuals from 7 to 1449 days post-H5N1 infection. All individuals whose sera was confirmed positive by an independent assay (WHO verification and/or Serology) were also positive using the diagnostic assays provided herein, indicating a low false negative rate. Only one individual that tested positive using the diagnostic peptides of the present invention was negative by the WHO culture test, but was confirmed to be infected by H5N1 using PCR based RNA detection. Importantly, the data also shows that the diagnostic assay of the present invention can detect antibodies to H5N1 within 7 days and up to 4 years post-infection (Table 1).

TABLE 1
Detection of H5N1 infection of individuals
in Vietnam using H5N1 serodiagnostic assay.

| Day Since | | | WHO | Serology |
|---|---|---|---|---|
| Onset | SelectAbTest | Outcome | verification | (HI + MA)* |
| 1449 | Positive | Survived | culture+ | |
| 1336 | Positive | Survived | culture neg. | |
| 1329 | Positive | Survived | culture+ | |
| 1310 | Positive | Survived | NIID culture+ | Positive |
| 1303 | Positive | Survived | CDC & NIID | Positive |
| | | | culture+ | |
| 1300 | Positive | Survived | culture+ | Positive |
| 1271 | Positive | Survived | culture+ | Negative |
| 1309 | Positive | Survived | | |
| 1309 | Positive | Survived | | |
| 1299 | Positive | Survived | | |
| 1277 | Positive | Survived | Negative CDC | |
| | | | & NIID | |
| | | | culture+ | |
| 1212 | Positive | Survived | culture+ | |
| 1255 | Positive | Survived | culture+ | |
| 1071 | Positive | Survived | | |
| 11 | Positive | Survived | NIHE culture | Positive |
| | | | +ve | |
| 13 | Positive | Survived | NIHE culture | Positive |
| | | | +ve | |
| 14 | Positive | Survived | NIHE culture | Positive |
| | | | +ve | |
| 15 | Positive | Survived | NIHE culture | Positive |
| | | | +ve | |
| 18 | Positive | Survived | NIHE culture | Positive |
| | | | +ve | |
| 21 | Positive | Survived | NIHE culture | Positive |
| | | | +ve | |
| 26 | Positive | Survived | NIHE culture | Positive |
| | | | +ve | |
| 62 | Positive | Survived | NIHE culture | Positive |
| | | | +ve | |
| 540 | Positive | Survived | NIHE culture | Positive |
| | | | +ve | |
| 13 | Positive | Survived | NIHE culture | Positive |
| | | | +ve | |
| 15 | Positive | Survived | NIHE culture | Positive |
| | | | +ve | |
| 18 | Positive | Survived | NIHE culture | Positive |
| | | | +ve | |
| 36 | Positive | Survived | NIHE culture | Positive |
| | | | +ve | |
| 45 | Positive | Survived | NIHE culture | Positive |
| | | | +ve | |
| 64 | Positive | Survived | NIHE culture | Positive |
| | | | +ve | |
| 535 | Positive | Survived | NIHE culture | Positive |
| | | | +ve | |
| 15 | Positive | Died | NIHE culture | Positive |
| | | | +ve | |
| 18 | Positive | Died | NIHE culture | Positive |
| | | | +ve | |
| 7 | Positive | Survived | | Positive |
| 10 | Positive | Survived | | Positive |
| 7 | Positive | Died | NIHE culture | |
| | | | +ve | |
| 8 | Positive | Died | NIHE culture | |
| | | | +ve | |
| 10 | Positive | Died | NIHE culture | |
| | | | +ve | |
| 84 | Positive | Survived | NIHE culture | |
| | | | +ve | |
| 9 | Positive | Died | NIHE culture | |
| | | | +ve | |
| 10 | Positive | Died | NIHE culture | |
| | | | +ve | |
| 11 | Positive | Died | NIHE culture | |
| | | | +ve e | |

*HI = Haemagglutination inhibition assay; MN = microneutralization assay.

This example demonstrates that the serodiagnostic assay of the present invention has a low false negative and false positive rate, and is able to detect seroconversions within 7 days and up to 4 years post-infection. Therefore, the assays provided herein are useful for serodiagnosis and surveillance of H5N1 infections around the world not only in humans but also in poultry and domestic animals as well as surveillance in wild birds and animals.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for detecting the presence, absence, or quantity of H5N1 antibodies in a sample, the method comprising,
(a) contacting the sample to:
a first polypeptide comprising a sequence at least 80% identical to SEQ ID NO:1, wherein the first polypeptide does not comprise more than 20 additional contiguous H5N1 Hemagglutinin amino acids on the amino terminus and the first polypeptide does not comprise more than five additional contiguous H5N1 Hemagglutinin amino acids on the carboxyl terminus; and
a second polypeptide comprising a sequence at least 80% identical to SEQ ID NO:2, optionally with one or more amino acids of SLLTE (SEQ ID NO:14) linked to the amino terminus, wherein the second polypeptide does not comprise more than five additional contiguous H5N1 M2e amino acids at either terminus; and
(b) detecting the presence, absence, or quantity of binding of at least one of the polypeptides to an antibody in the sample, thereby detecting the presence or absence of H5N1 antibodies in the sample.

2. The method of claim 1, wherein the contacting step (a) further comprises contacting the sample to a third polypeptide comprising a sequence at least 80% identical to SEQ ID NO:3, wherein the third polypeptide does not comprise more than five additional contiguous H5N1 PB1-F2 amino acids at either terminus, and the detecting step (b) further comprises detecting the presence or absence of binding of the third polypeptide to an antibody in the sample.

3. (canceled)

4. The method of claim 2, wherein the first polypeptide comprises SEQ ID NO:1 and/or the second polypeptide comprises SEQ ID NO:2 and/or the third polypeptide comprises SEQ ID NO:3.

5. The method of claim 2, wherein the first polypeptide consists of SEQ ID NO:1 and/or the second polypeptide consists of SEQ ID NO:2 and/or the third polypeptide consists of SEQ ID NO:3.

6. The method of claim 1, wherein the second polypeptide comprises SEQ ID NO:8.

7. The method of claim 1, wherein the first polypeptide comprises, or consists of, SEQ ID NO:9.

8. The method of claim 1, wherein the second polypeptide comprises, or consists of, SEQ ID NO:10.

9. The method of claim 2, wherein the third polypeptide comprises, or consists of, SEQ ID NO:11.

10. (canceled)

11. (canceled)

12. The method of claim 1, wherein the sample is from a human.

13. The method of claim 1, wherein the sample is from a non-human animal.

14. The method of claim 1, wherein the sample is from a bird.

15. A kit for detecting the presence or absence of H5N1 antibodies in a sample, the kit comprising:
a first polypeptide comprising a sequence at least 80% identical to SEQ ID NO:1, wherein the first polypeptide does not comprise more than 20 additional contiguous H5N1 Hemagglutinin amino acids on the amino terminus and the first polypeptide does not comprise more than five additional contiguous H5N1 Hemagglutinin amino acids on the carboxyl terminus; and
a second polypeptide comprising a sequence at least 80% identical to SEQ ID NO:2, optionally with one or more amino acids of SLLTE (SEQ ID NO:14) linked to the amino terminus, wherein the second polypeptide does not comprise more than five additional contiguous H5N1 M2e amino acids at either terminus,
wherein the first and second polypeptides are linked to one or more solid supports.

16. The kit of claim 15, wherein the kit further comprises a third polypeptide comprising a sequence at least 80% identical to SEQ ID NO:3, wherein the third polypeptide does not comprise more than five additional contiguous H5N1 PB1-F2 amino acids at either terminus.

17. (canceled)

18. The kit of claim 16, wherein the first polypeptide comprises SEQ ID NO:1 and/or the second polypeptide comprises SEQ ID NO:2 and/or the third polypeptide comprises SEQ ID NO:3.

19. The kit of claim 16, wherein the first polypeptide consists of SEQ ID NO:1 and/or the second polypeptide consists of SEQ ID NO:2 and/or the third polypeptide consists of SEQ ID NO:3.

20. The kit of claim 15, wherein the second polypeptide comprises SEQ ID NO:8.

21. The kit of claim 15, wherein the first polypeptide comprises, or consists of, SEQ ID NO:9.

22. The kit of claim 15, wherein the second polypeptide comprises, or consists of, SEQ ID NO:10.

23. The kit of claim 16, wherein the third polypeptide comprises, or consists of, SEQ ID NO:11.

24. (canceled)

25. (canceled)

26. The kit of claim 16, wherein the first and second, and if included in the kit, the third polypeptides are linked to a solid surface.
